(19) United States
(12) Patent Application Publication
(10) Pub. No.: US 2015/0176070 A1
(43) Pub. Date: Jun. 25, 2015

(54) FLOW CELL FOR BIOMATERIAL ANALYSIS AND BIOMATERIAL ANALYSIS DEVICE

(71) Applicant: HITACHI HIGH-TECHNOLOGIES CORPORATION, Tokyo (JP)

(72) Inventors: Yuichiro Ota, Tokyo (JP); Tomohiro Shoji, Tokyo (JP); Michiru Fujioka, Tokyo (JP); Takanobu Haga, Tokyo (JP)

(21) Appl. No.: 14/414,235

(22) PCT Filed: Jul. 11, 2013
(86) PCT No.: PCT/JP2013/069053

§ 371 Date: Jan. 12, 2015

(30) Foreign Application Priority Data
Jul. 13, 2012 (JP) 2012-157625

(51) Int. Cl. C12Q 1/68; G01N 21/03; G01N 15/14; G01N 21/64

(52) U.S. Cl. CPC C12Q 1/6869; G01N 21/6428; G01N 21/645; G01N 21/0332; G01N 15/1459; G01N 2021/6482; G01N 2201/061; G01N 2201/0231

(57) ABSTRACT

In a biomaterial analysis, erroneous detection of a particle emitting fluorescence is prevented, and highly sensitive and highly accurate optical detection in biomaterial analysis is performed. A flow cell (104) for biomaterial analysis includes: a light-transmissive upper substrate (310); an antireflective lower substrate (313); and an inner layer section interposed between the upper substrate (310) and the lower substrate (313) and including a flow path (311) in which a particle (312) configured to emit fluorescence is provided. A biomaterial analysis device includes: a flow cell (104) for biomaterial analysis as described above; and an irradiation unit configured to irradiate excitation light; and an optical detection unit (106) configured to detect fluorescence emitted by the particle (312).

### TECHNICAL FIELD

The present invention relates to a flow cell for biomaterial analysis and a biomaterial analysis device.

### BACKGROUND ART

A new technology to determine a DNA or RNA base sequence has recently been developed. There has been proposed a method in which a large number of DNA fragments to be analyzed are fixed to a substrate and base sequences of such DNA fragments are determined in parallel.

In Non-patent Document 1, particles are used as a carrier for carrying DNA fragments and PCR (polymerase chain reaction) is performed on the particles. Then, particles carrying PCR-amplified DNA fragments are introduced into a plate provided with many holes whose diameter coincides with the size of the particles, and base sequences are read using a pyrosequencing method.

In Non-patent Document 2, particles are used as a carrier for carrying DNA fragments and PCR is performed on the particles. Then, the particles are scattered and fixed on a glass substrate, and an enzyme reaction (ligation) is carried out on the glass substrate. Thereafter, fluorescence is detected by taking in a substrate with a fluorescent pigment. Thus, sequence information on each of the fragments is obtained. In the method disclosed in Non-patent Document 2, the glass substrate is used as a flow cell.

Here, the flow cell has a flow path in one or more channels and also has a spacer bonded or welded in a sandwiched manner between glass substrates. The particles carrying the DNA fragments are attached on an inner wall of the flow path in the flow cell. An area including tens of thousands of to hundreds of thousands of the particles is collectively irradiated with excitation light, and fluorescence emitted from the tens of thousands of to hundreds of thousands of particles (to be precise, fluorescence emitted from fluorescent pigments taken into the DNA fragments fixed to the particles) is detected all at once with one camera. A detection optical system including the camera has an optical resolution and a function to measure light intensity, which make it possible to specify the position of each of the particles. The detection optical system detects where the particles are located and how intense is the light emitted from those particles.

As described above, there has been developed and put to practical use a method for determining sequence information of many fragments in parallel by fixing many nucleic acid fragment samples on a substrate.

Patent Documents 1 to 3 disclose a method for analyzing a base sequence of a DNA fragment by using a microfluid device having DNA fragments fixed to various carriers.

### PRIOR ART DOCUMENT

### Patent Document

Patent Document 1: Japanese Patent Application Publication No. 2005-224110
Patent Document 2: Japanese Patent Application Publication No. 2005-130795
Patent Document 3: Japanese Patent Application Publication No. 2004-333255

### Non-Patent Document

Non-patent Document 1: Marcel Margulies et al., Nature, 2005, vol. 437, P376-380
Non-patent Document 2: Jay Shendure et al., Science, 2005, vol. 309, P1728-1732

### SUMMARY OF THE INVENTION

### Problem to be Solved by the Invention

In DNA base sequence analysis using the flow cell, it is required to accurately distinguish fluorescence emitted from particles binding individually different DNA fragments provided on the flow cell and to simultaneously detect the positions, colors and light intensities thereof. There can be tens of thousands of particles, and extremely high measurement accuracy is required for detection of fluorescence emitted from those particles. However, because of the structure of the flow cell, lights emitted from adjacent particles interfere with each other. Thus, it has been found out that there is a problem that the accuracy of DNA base sequence analysis is not increased more than a certain level.

The present invention is made in consideration of the foregoing circumstances. It is an object of the present invention to prevent erroneous detection of particles emitting fluorescence, and enables highly sensitive and highly accurate optical detection in biomaterial analysis.

### Solution to the Problem

The inventors of the present invention have conducted a keen study to find out why the analysis accuracy of fluorescence emitted from tens of thousands of particles provided on the flow cell is not increased more than a certain level. As a result, the inventors of the present invention have reached the present invention by finding out that light emitted from particles adjacent to particles to be analyzed is reflected by an interface between a lower substrate included in the flow cell and an external air layer and is mixed with light emitted from the particles to be analyzed, causing an analysis error.

A flow cell for biomaterial analysis according to the present invention includes: a light-transmissive upper substrate; an antireflective lower substrate; and an inner layer section interposed between the upper substrate and the lower substrate and including a flow path in which a particle configured to emit fluorescence is provided. A flow cell for biomaterial analysis according to the present invention includes: a light-transmissive upper substrate; a light-transmissive lower substrate; and an inner layer section interposed between the upper substrate and the lower substrate, and the inner layer section includes: a flow path in which a particle configured to emit fluorescence is provided; and an antireflective spacer. A biomaterial analysis device according to the present invention includes: a flow cell for biomaterial analysis as described above; an irradiation unit configured to irradiate excitation light; and an optical detection unit configured to detect fluorescence emitted by the particle.

### Advantageous Effects of the Invention

The present invention prevents erroneous detection of particles emitting fluorescence, and enables highly sensitive and highly accurate optical detection in biomaterial analysis.

### BRIEF DESCRIPTION OF THE DRAWINGS

FIG. 1 is a schematic diagram of a biomaterial analysis device according to an embodiment of the present invention.

FIG. 2 is a diagram showing how a flow cell for biomaterial analysis is attached to the biomaterial analysis device according to the embodiment of the present invention.

FIG. 3 is a schematic partial cross-sectional view of the flow cell for biomaterial analysis and the biomaterial analysis device according to the embodiment of the present invention.

FIG. 4A is a schematic partial cross-sectional view of the flow cell for biomaterial analysis according to the embodiment of the present invention.

FIG. 4B is a partially enlarged view of FIG. 4A.

FIG. 5A is a schematic partial cross-sectional view of a flow cell for biomaterial analysis according to a comparative example.

FIG. 5B is a partially enlarged view of FIG. 5A.

FIG. 6 is a diagram showing a configuration of the flow cell for biomaterial analysis according to the embodiment of the present invention.

### EMBODIMENTS FOR CARRYING OUT THE INVENTION

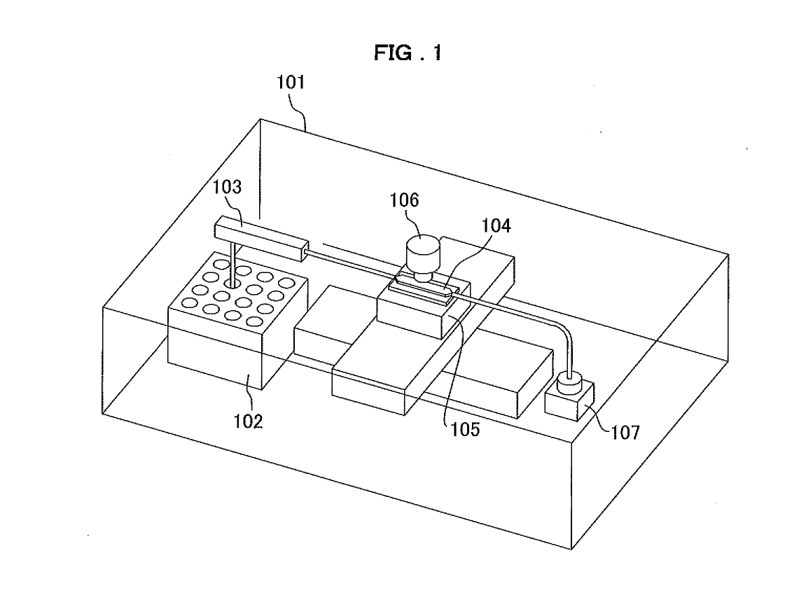

With reference to the drawings, embodiments of the present invention are described in detail below. Note that the embodiments of the present invention are not limited to the following embodiments. However, contents shown in FIGS. 1 to 3 and description regarding FIGS. 1 to 3 to be given later are common to the embodiments and a comparative example of the present invention.

In the present invention, a biomaterial means a chemical substance that expresses some kind of functions within a biological body such as nucleic acid such as DNA and RNA, protein, peptide, antibody and antigen. Particularly, the biomaterial means a chemical substance that has a high-order structure in which a large number of chemical substances, each serving as a unit, are connected, and exerts functions as the whole biomaterial by the arrangement of the chemical substances as individual units. In the present invention, nucleic acid particularly has the aptitude among such biomaterials.

Various biomaterial analyses are performed using a flow cell for biomaterial analysis and a biomaterial analysis device according to the present invention. For example, determination of DNA arrangement (DNA sequence) and hybridization can be performed.

With reference to FIG. 1, description is given of an overview of the biomaterial analysis device according to the embodiment of the present invention. Here, the description is given using nucleic acid as a target biomaterial.

A biomaterial analysis device (nucleic acid analysis device) 101 according to the embodiment of the present invention includes: a reagent cooling and storage box 102 for housing reagent containers and the like; a liquid sending mechanism 103 for sending a reaction liquid in each of the reagent containers; a flow cell 104 having a flow path in which particles binding DNA fragments are provided; a temperature control substrate 105 configured to control the temperature of the flow path in the flow cell 104; and an optical detection unit 106 configured to detect fluorescence emitted by fluorescent substances taken into the DNA fragments bound to the particles. A reaction liquid for nucleic acid analysis is supplied to the preadjusted flow path in the flow cell 104 by the liquid sending mechanism 103. The reaction liquid causes air elongation reaction on the particles in the flow cell 104, and the fluorescent substances taken into the DNA fragments emit the fluorescence. The optical detection unit 106 detects the emitted fluorescence for base sequence analysis. Excess reaction liquid, cleaning liquid and the like after the reaction are housed in a waste tank 107.

In the embodiment of the present invention, examples of a DNA sequence analysis method include, but not particularly limited to, one using stepwise ligation (Sequencing by Oligonucleotide Ligation and Detection). The stepwise ligation is a method for sequentially binding fluorescently-labeled probes by using single-stranded DNA on the particles as a template and then determining a sequence for every two bases. As an enzyme reaction caused by ligase, oligonucleotides including a fluorescent portion corresponding to a target sequence of the DNA fragments are bound to cause the elongation reaction. After the completion of the reaction, the fluorescent portion is irradiated with four colors of excitation light, and the cal detection unit detects the fluorescence. Thereafter, the fluorescent portion is cut off, and then a further elongation reaction is carried out to detect the fluorescence corresponding to the next sequence. By eating the above operation, base sequences corresponding to four fluorescent colors are determined one after another, resulting in a base sequence of the DNA fragments. This method enables sequencing of several tens to hundreds of by per cycle, and enables data analysis of several tens of Gb per run. In the stepwise ligation, fluorescently-labeled oligonucleotides are repeatedly hybridized, thereby enabling parallel sequencing.

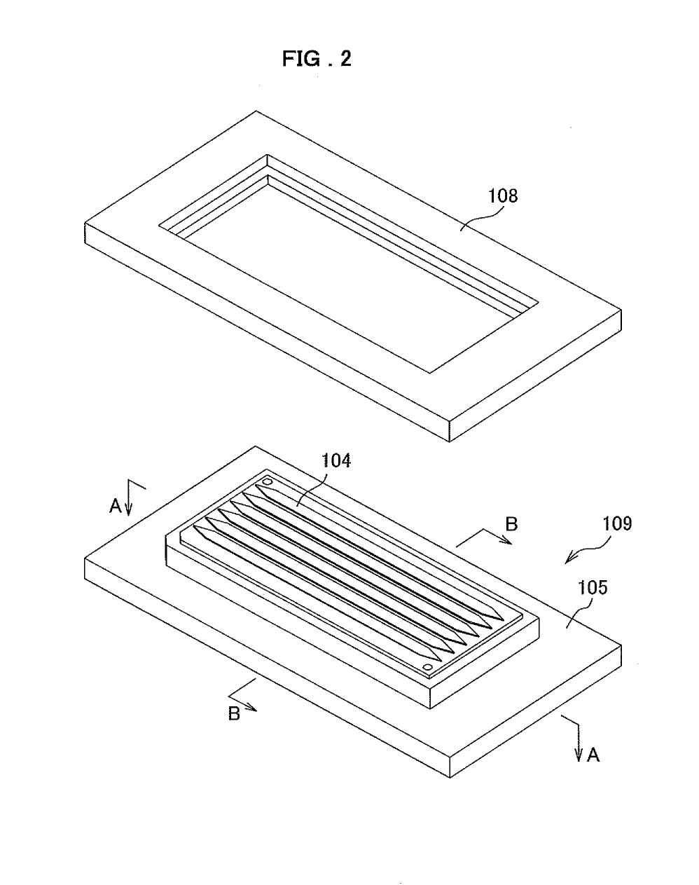

FIG. 2 is a diagram showing how the flow cell for biomaterial analysis is attached to the biomaterial analysis device according to the embodiment of the present invention. The flow cell 104 for biomaterial analysis (hereinafter may be called the “flow cell”) is placed on the temperature control substrate 105 with high flatness and fitted to an attachment part 109 in the biomaterial analysis device by pressing a cover 108 against the attachment part. A large number of the flow cells 104 can be arranged in parallel. In FIG. 2, six flow cells 104 are arranged in parallel.

Moreover, in order to facilitate a reaction between the DNA fragments and the reaction liquid, it is preferable that the biomaterial analysis device includes the temperature control substrate 105 to control the temperature of the flow path in each of the flow cells 104.

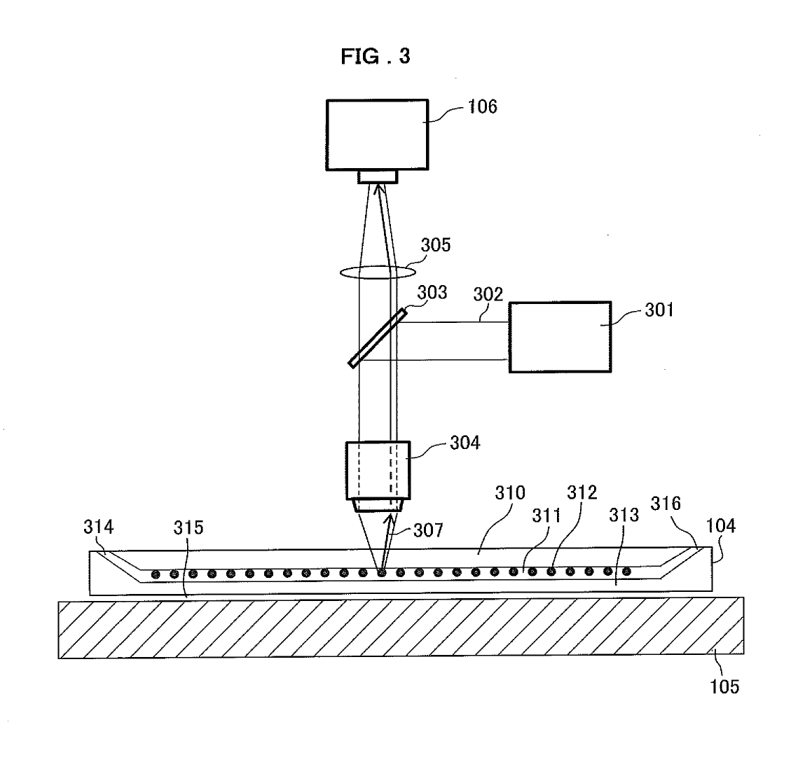

FIG. 3 is a schematic partial cross-sectional view of the flow cell for biomaterial analysis and the biomaterial analysis device according to the embodiment of the present invention. FIG. 3 is a cross-sectional view taken along the line A-A in FIG. 2.

An irradiation unit for irradiating excitation light includes a lamp 301, a mirror 303 and an objective lens 304. An excitation light 302 emitted from the lamp 301 to excite the fluorescent substances is reflected by the mirror 303. The excitation light 302 passes through the objective lens 304 after reflected and is irradiated from above onto particles 312 provided in a flow path 311 in an inner layer section of the flow cell 104 placed on the temperature control substrate 105. The fluorescent substances present on the surfaces of the particles 312 are excited by the excitation light 302 to emit fluorescence 307. The fluorescence 307 passes through the objective lens 304 thereabove and passes through the mirror 303 and the lens 305 before reaching a camera included in the optical detection unit 106 to detect the fluorescence emitted by the particles 312.

The flow cell 104 includes a light-transmissive upper substrate 310, a lower substrate 313 and the inner layer section interposed between the upper substrate 310 and the lower substrate 313 and having the flow path 311 in which the particles 312 configured to emit the fluorescence are provided. The flow cell 104 includes an inlet 314, from which the reaction liquid is introduced, and an outlet 316, from which the reaction liquid is discharged, at both ends of the flow path 311 in the inner layer section.

The particles 312 emitting the fluorescence are provided inside the flow path 311 in the inner layer section of the flow cell 104, and may be provided in any position as long as the optical detection unit 106 can detect the fluorescence emitted by the particles 312. In FIG. 3, the particles 312 emitting the fluorescence are provided in the upper part of the flow path 311 in the inner layer section of the flow cell 104, i.e., below the lower surface of the upper substrate 310.

There is also a gap, i.e., an air layer 315 between the flow cell 104 and the temperature control substrate 105. This is because it is difficult to manufacture the device in a state where the lower surface of the flow cell 104 completely coincides with the upper surface of the temperature control substrate 105.

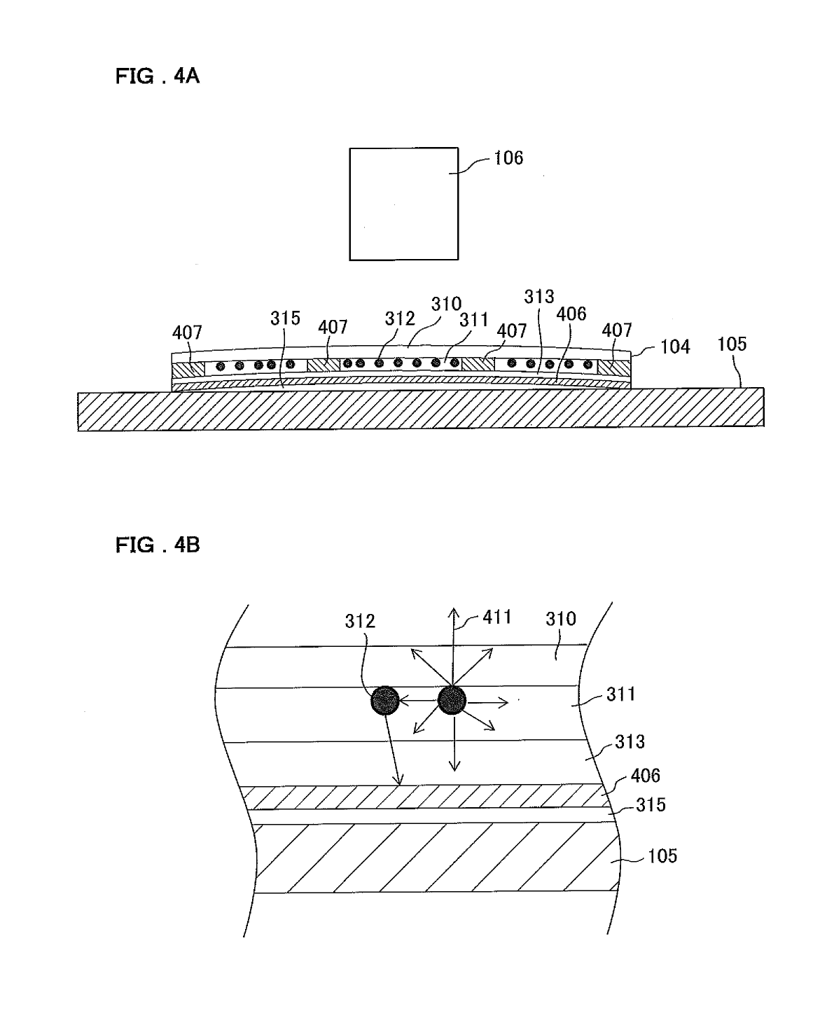

FIG. 4A is a schematic partial cross-sectional view of the flow cell for biomaterial analysis according to the embodiment of the present invention. FIG. 4B is a partially enlarged view of FIG. 4A. FIGS. 4A and 4B are both cross-sectional views taken along the line B-B in FIG. 2. While the six flow cells 104 are arranged in parallel in FIG. 2, FIG. 4A shows only three of those.

In FIG. 4A, as in the case of FIG. 3, the flow cell 104 includes the light-transmissive upper substrate 310, the lower substrate 313 and the inner layer section interposed between the upper substrate 310 and the lower substrate 313 and having the flow path 311 in which the particles 312 configured to emit the fluorescence are provided. Both ends of the flow path 311 are hermetically sealed by spacers 407. Since the flow cell 104 is somewhat curved, there is a gap, i.e., the air layer 315 between the flow cell 104 and the temperature control substrate 105. Furthermore, in the embodiment of the present invention, there is an antireflective material layer 406 on the air layer 315-side surface of the lower substrate 313.

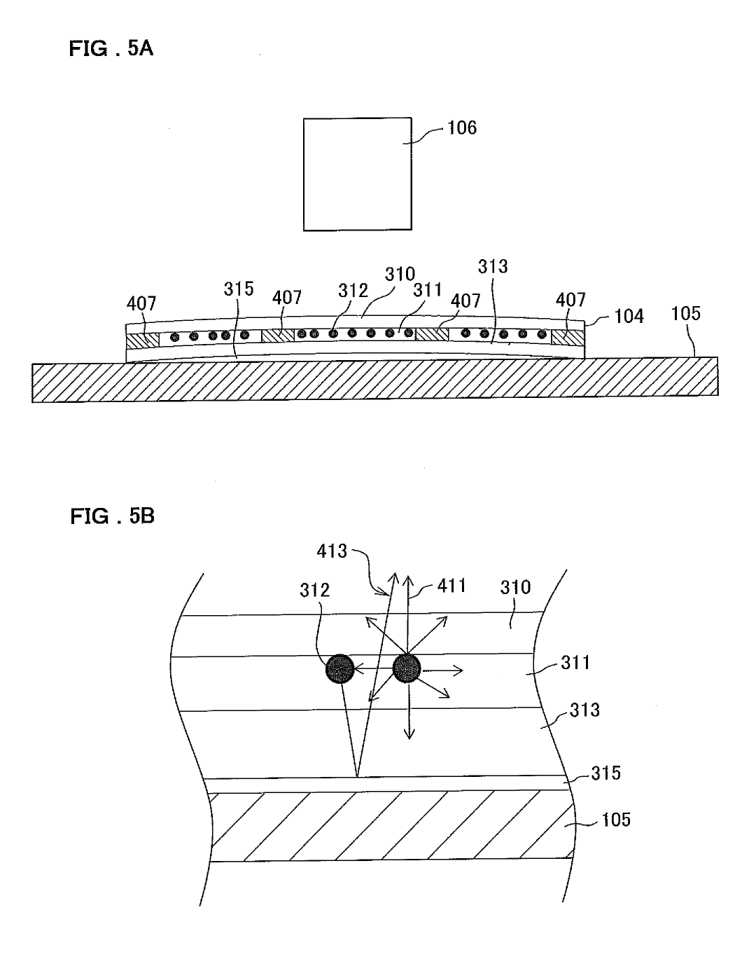

FIG. 5A is a schematic partial cross-sectional view of a flow cell for biomaterial analysis according to a comparative example. FIG. 5B is a partially enlarged view of FIG. 5A. FIGS. 5A and 5B are both cross-sectional views taken along the line B-B in FIG. 2. While the six flow cells 104 are arranged in parallel in FIG. 2, FIG. 5A shows only three of those.

In FIG. 5A, as in the case of FIG. 4A, the flow cell 104 includes a light-transmissive upper substrate 310, a lower substrate 313 and an inner layer section interposed between the upper substrate 310 and the lower substrate 313 and having a flow path 311 in which particles 312 configured to emit fluorescence are provided. Both ends of the flow path 311 are hermetically sealed by spacers 407. Since the flow cell 104 is somewhat curved, there is a gap, i.e., an air layer 315 between the flow cell 104 and the temperature control substrate 105.

The comparative example is described with reference to FIG. 5B.

When the excitation light is irradiated onto the fluorescent substances taken into the DNA fragments carried by the particles 312 provided inside the flow path 311 in the inner layer section of the flow cell 104, fluorescence 411 is emitted radially in all directions from the particles 312.

Some of the emitted fluorescence 411 passes through the light-transmissive upper substrate 310 and is received by the optical detection unit 106 to detect the fluorescence emitted by the particles 312. The rest of the emitted fluorescence 411 travels through the lower substrate 313 and is reflected by an interface with the air layer 315 between the lower substrate 313 and the temperature control substrate 105. Some fluorescence 413 reflected by an interface between the lower substrate 313 and the air layer 315 is received by the optical detection unit 106 after passing through an optical path different from those described above.

When an optical signal detected by the optical detection unit 106 is observed as a two-dimensional image, an optical signal of the fluorescence 413 reflected by the interface between the lower substrate 313 and the air layer 315 and then received by the optical detection unit 106 is observed such that the optical signal is generated from a position different from a position where the particles 312 as the original source of the fluorescence are located. Such an optical signal becomes background light (stray light), which hinders accurate detection of the position of the particles 312 emitting the fluorescence. Meanwhile, when the optical signal of the fluorescence 413 received by the optical detection unit 106 is observed such that the optical signal is generated from the particles 312 adjacent to the particles 312 which are the original source of the fluorescence (crosstalk), an analysis error occurs, which lowers reliability of the analysis. Such phenomena lower the accuracy of the biomaterial analysis device.

Such phenomena are particularly prominent when fluorescence intensity is low and detection is performed with higher sensitivity. The highly-sensitive optical detection unit 106 detects even a small amount of light, and thus reacts to the reflected light on the interface as described above, causing the entire background of the detected image to become bright. This increases a possibility that the particles 312 emitting a small amount of fluorescence are overlooked or the position of the particles 312 is erroneously detected.

With reference to FIG. 4B, the embodiment of the present invention is described.

When the excitation light is irradiated onto the fluorescent substances taken into the DNA fragments carried by the particles 312 provided inside the flow path 311 in the inner layer section of the flow cell 104, fluorescence 411 is emitted radially in all directions from the particles 312.

Some of the emitted fluorescence 411 passes through the light-transmissive upper substrate 310 and is received by the optical detection unit 106 to detect the fluorescence emitted by the particles 312. The rest of the emitted fluorescence 411 travels through the lower substrate 313 and enters or strikes on the antireflective material layer 406 on the air layer 315-side surface of the lower substrate 313 before disappearing or being absorbed. Here, a black coating film is formed as the antireflective material layer 406.

As a result, a phenomenon can be suppressed that some of the fluorescence emitted by the particles 312 is reflected by the interface with the air layer 315 between the lower substrate 313 and the temperature control substrate 105 and received by the optical detection unit 106. Accordingly, the accuracy of the biomaterial analysis device can be prevented from being lowered by the background light (stray light). More specifically, erroneous detection of the particles 312 emitting the fluorescence can be prevented to enable highly sensitive and highly accurate optical detection.

In the flow cell 104 for biomaterial analysis according to the embodiment of the present invention, the lower substrate 313 needs to be antireflective so that some of the fluorescence emitted by the particles 312 is not reflected by the interface with the air layer 315. In order for the lower substrate 313 to be antireflective, the lower substrate 313 may be a substrate made of an antireflective material or may be a substrate having the antireflective material layer 406. The antireflective material layer 406 may be located on the air layer 315-side surface of the lower substrate 313, may be located on the inner layer section-side surface of the lower substrate 313 or may be located on the inside between the both. Alternatively, more than one of those described above may be used in combination.

In formation of the antireflective material layer 406 in the lower substrate 313, a film or sheet made of an antireflective material may be laminated on the lower substrate 313, or printing ink or a coating agent may be applied onto the surface of the lower substrate 313. In formation of the antireflective material layer 406, it is important to form the layer without any gap so as not to form the air layer. Moreover, the antireflection property is advantageous for the flow path 311 in the flow cell 104. Thus, the antireflective material may be used only in a portion of the lower substrate 313 corresponding to the portion where the flow path 311 is located.

Here, the antireflection property means reduction in reflection of the fluorescence and the like emitted by the particles 312. To be more specific, it is preferable to use a material whose ratio of reflected light intensity to incident light is 50% or less, preferably 20% or less, more preferably 10% or less. Generally, most of the fluorescence used for the biomaterial analysis device such as the nucleic acid analysis device and emitted by the fluorescent substances taken into the DNA fragments is visible light. Therefore, a material antireflective to visible light is preferable. The visible light is light having a wavelength of about 400 nm to 700 nm.

The following are specific examples of the antireflective material effective in preventing the reflection of the visible light.

(1) black pigment or black dye; carbon black pigment such as carbon black and black lead, metal oxide black pigment such as ferrioxide, composite oxide of copper and chrome, composite oxide of copper, chrome and zinc, metal complex black dye, and the like.
(2) resin composition (thermosetting or thermoplastic) containing the above black pigment or black dye; black resin, paint, ink, coating agent, and the like. Specific examples of the resin include acrylic resin, cycloolefin polymer, and the like.
(3) glass containing the above black pigment or black dye, and the like.
(4) film of materials different in refractive index; a single-layer or multi-layer film is formed of materials different in refractive index, such as magnesium fluoride and metal oxide.

In formation of the antireflective material layer 406 in the lower substrate 313, glass, acrylic resin, polycycloolefin resin or the like, which is light transmissive material, can be used as the material of the lower substrate 313 other than the antireflective material layer 406, as in the case of the upper substrate 310 to be described later.

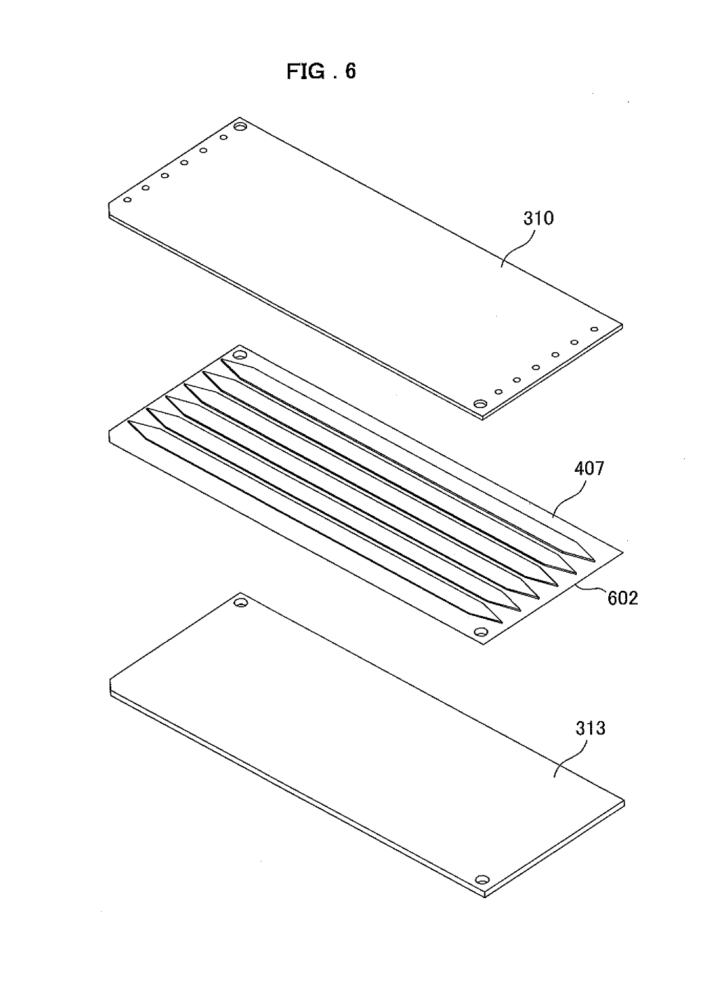

FIG. 6 is a diagram showing a configuration of the flow cell for biomaterial analysis according to the embodiment of the present invention.

The flow cell for biomaterial analysis according to the embodiment of the present invention includes the upper substrate 310, the lower substrate 313 and an inner layer section 602 interposed between the upper substrate 310 and the lower substrate 313.

The inner layer section 602 includes; a flow path in which particles configured to emit fluorescence are provided; and a spacer 407 around the flow path to prevent leakage of a reaction liquid to be supplied to the flow path.

The upper substrate 310 is light-transmissive. Here, the light transmissivity means permeability to fluorescence emitted by the particles and the like to enable detection by the optical detection unit 106. Generally, most of the fluorescence used for the biomaterial analysis device such as the nucleic acid analysis device and emitted by the fluorescent substances taken into the DNA fragments is visible light. Therefore, it is preferable that the upper substrate 310 is transmissive to visible light. Examples of the light transmissive material that can be used as the upper substrate 310 include glass, acrylic resin, polycycloolefin resin, and the like.

The upper substrate 310 needs to have excellent light transmissivity, and thus is preferably formed to have a small thickness. On the other hand, the lower substrate 313 preferably has a certain thickness and mechanical strength, in consideration of handleability as the flow cell 104. Therefore, it is preferable that the thickness of the upper substrate 310 is equal to or smaller than that of the lower substrate 313.

The following are examples of a method for manufacturing a flow cell having a flow path.

(i) interposing a sheet between the upper substrate 310 and the lower substrate 313, the sheet having a hollow in a portion corresponding to the flow path and including only the spacer 407 around the flow path.
(ii) removing a portion of the upper substrate 310 or the lower substrate 313, the portion corresponding to the flow path.

In the above method (i), in order to form the flow path in which particles configured to emit fluorescence are provided, the particles configured to emit fluorescence are provided beforehand in the portion of the upper substrate 310 or the lower substrate 313 corresponding to the flow path. For detection of the fluorescence emitted by the particles with higher accuracy, the particles are preferably provided on the lower surface of the upper substrate 310.

The height of the flow path 311, i.e., the thickness of the spacer 407 is preferably 1 mm or less for accurate temperature control. Moreover, it is preferable that the flow path 311 in the flow cell 104 has one or more inlets 314 and outlets 316 for the reaction liquid.

In the embodiment of the present invention, as the particles 312 emitting the fluorescence, the particles 312 binding the DNA fragments and the like can be prepared beforehand and fixed onto the upper substrate 310 or the lower substrate 313 with a fixing agent or the like. Alternatively, carriers binding the DNA fragments and the like can be directly fixed in a particulate pattern onto the upper substrate 310 or the lower substrate 313 without preparing the particles 312 beforehand. Alternatively, the DNA fragments and the like can be directly fixed in a dot pattern onto the upper substrate 310 or the lower substrate 313.

When the particles 312 emitting the fluorescence are provided on the upper substrate 310 or the lower substrate 313, it is preferable to provide a fixation layer made of inorganic oxide beforehand on the substrate, in order to fix the prepared particles, particulate carriers, DNA fragments or the like described above. By providing the fixation layer, the particles, particulate carriers, DNA fragments or the like can be more firmly fixed onto the substrate. The inorganic oxide can be selected from the group including titania, zirconia, alumina, zeolite, vanadium pentoxide, silica, sapphire, tungsten oxide, tantalum pentoxide, and a composite of at least two of the above.

A second embodiment of the present invention is described below, which is different from the embodiment of the present invention described above. The second embodiment of the present invention uses a flow cell for biomaterial analysis (not shown), including a light-transmissive upper substrate, a light-transmissive lower substrate and an inner layer section interposed between the upper substrate and the lower substrate and having a flow path in which particles configured to emit fluorescence are provided, and an antireflective spacer.

In the second embodiment of the present invention, both of the upper and lower substrates are light transmissive, excitation light is irradiated from above the flow cell, and fluorescence emitted by the particles is detected by an optical detection unit below the flow cell. Thus, some of the fluorescence emitted by the particles in the flow path is reflected by an interface between the lower substrate and an air layer. Accordingly, such fluorescence is less likely to hinder the analysis.

However, some of the fluorescence emitted by the particles may be leaked to the spacer and detected by the optical detection unit below the flow cell, resulting in an analysis error. Therefore, since the spacer is antireflective, some of the fluorescence entering the spacer is reflected, enabling prevention of measurement errors.

### EXPLANATION OF REFERENCE NUMERALS

101 biomaterial analysis device (nucleic acid analysis device)
102 reagent cooling and storage box
103 liquid sending mechanism
104 flow cell
105 temperature control substrate
106 optical detection unit
107 waste tank
108 cover
109 attachment pa
301 lamp
310 upper substrate
311 flow path
312 particles
313 lower substrate
315 air layer
406 antireflective material layer
407 spacer
602 inner layer section

1. A flow cell for biomaterial analysis, comprising:
a light-transmissive upper substrate;
an antireflective lower substrate; and
an inner layer section interposed between the upper substrate and the lower substrate and including a flow path in which a particle configured to emit fluorescence is provided.

2. The flow cell for biomaterial analysis, according to claim 1,
wherein the lower substrate is a substrate made of an antireflective material or a substrate including an antireflective material layer.

3. The flow cell for biomaterial analysis, according to claim 1,
wherein a thickness of the upper substrate is equal to or smaller than a thickness of the lower substrate.

4. The flow cell for biomaterial analysis, according to claim 1,
wherein the particle is provided on a lower surface of the upper substrate.

5. The flow cell for biomaterial analysis, according to claim 1,
wherein the biomaterial includes a nucleic acid.

6. The flow cell for biomaterial analysis, according to claim 5,
wherein a nucleic acid fragment is bound to the particle.

7. The flow cell for biomaterial analysis, according to claim 5,
wherein a reaction liquid for nucleic acid analysis is supplied to the flow path.

8. A biomaterial analysis device comprising:
a flow cell for biomaterial analysis according to claim 1;
an irradiation unit configured to irradiate excitation light; and
an optical detection unit configured to detect fluorescence emitted by the particle.

9. The biomaterial analysis device according to claim 8, further comprising:
a temperature control substrate configured to control the temperature of the flow path in the flow cell for biomaterial analysis.

10. A flow cell for biomaterial analysis, comprising:
a light-transmissive upper substrate;
a light-transmissive lower substrate; and
an inner layer section interposed between the upper substrate and the lower substrate, and
wherein the inner layer section includes: a flow path in which a particle configured to emit fluorescence is provided; and an antireflective spacer.

11. The flow cell for biomaterial analysis, according to claim 10,
wherein a thickness of the upper substrate is equal to or smaller than a thickness of the lower substrate.

12. The flow cell for biomaterial analysis, according to claim 10,
wherein the particle is provided on a lower surface of the upper substrate.

13. The flow cell for biomaterial analysis, according to claim 10,
wherein the biomaterial includes a nucleic acid.

14. The flow cell for biomaterial analysis, according to claim 13,
wherein a nucleic acid fragment is bound to the particle.

15. The flow cell for biomaterial analysis, according to claim 13,
wherein a reaction liquid for nucleic acid analysis is supplied to the flow path.

16. A biomaterial analysis device comprising:
a flow for biomaterial analysis according to claim 10;
an irradiation unit configured to irradiate excitation light; and
an optical detection unit configured to detect fluorescence emitted by the particle.

17. The biomaterial analysis device according to claim 16, further comprising:
a temperature control substrate configured to control the temperature of the flow path in the flow cell for biomaterial analysis.
